(19) United States
(12) Patent Application Publication
(10) Pub. No.: US 2025/0236583 A1
(43) Pub. Date: Jul. 24, 2025

(54) CATALYST AND METHODS FOR OLEFIN ACETOXYLATION

(71) Applicant: Celanese International Corporation, Irving, TX (US)

(72) Inventors: Steven R. Alexander, Irving, TX (US); Laiyuan Chen, Irving, TX (US); Sean Mueller, Irving, TX (US)

(73) Assignee: Celanese International Corporation, Irving, TX (US)

(21) Appl. No.: 18/421,643

(22) Filed: Jan. 24, 2024

(51) Int. Cl. C07C 67/055; B01J 21/08; B01J 21/12; B01J 23/52; B01J 35/51

(52) U.S. Cl. CPC C07C 67/055; B01J 21/08; B01J 21/12; B01J 23/52; B01J 35/51

(57) ABSTRACT

Disclosed herein catalyst, a catalyst substrate, a catalyst and catalyst substrate shape, and a process of acetoxylating olefins using the same. The catalyst can comprise a support material having a catalytic layer disposed thereon and having a pimpled and/or dimpled shape as defined herein. The catalytic layer can comprise palladium and gold.

### FIELD OF THE INVENTION

The invention relates to a catalyst, a catalyst substrate, a catalyst and catalyst substrate shape, and to processes of acetoxylating olefins using the same. For example, the invention relates to a catalyst comprising a support material having a catalytic layer disposed thereon and having a pimpled and/or dimpled shape as defined herein. In some embodiments, the catalytic layer can comprise palladium and gold, for example in a concentration of 7 to 10 g/L palladium and from 3 to 8 g/L gold. In some embodiments, the catalyst has an average diameter, as measured by the longest diameter of the catalyst, of from 4 mm to 8 mm (e.g., from 4 mm to 7 mm).

### BACKGROUND OF THE INVENTION

Processes for the acetoxylation of olefins are of great economic importance. Processes for the acetoxylation of ethylene in the gas phase to form high yields of vinyl acetate monomer are of particular industrial interest. Acetoxylations may be carried out industrially by catalytic gas-phase oxidation of olefins, such as ethylene or propylene, in fixed-bed reactors. These reactions may be carried out in shell-and-tube reactors. Unsaturated esters, such as vinyl acetate, may be prepared by this reaction. In general, the reaction is carried out by passing a gaseous mixture comprising molecular oxygen, an olefin and acetic acid through a reactor. The reaction is typically conducted in a shell-and-tube reactor in which a plurality of reaction tubes are arranged in parallel and in each of which a uniform catalyst charge is located. The excess heat of reaction involved is removed by means of a heat transfer medium. An example of such a reactor is a boiling water reactor.

Pressure drop of the reactor feed gases flowing through a vinyl acetate reactor limits the total volume of gases that can be fed to the reactor. This, in turn, also places limits on the maximum obtainable mass transfer and heat transfer effects in the reactor bed to drive the reaction kinetics at the surface of the catalyst and to remove products and reaction heat from the system. Pressure drop through a packed reactor bed can be described by the Ergun equation, with a key variable being the void fraction of the packed catalyst bed.

Efforts to reduce pressure drop in the above process are described in various references.

U.S. Pat. No. 6,399,813 discloses a highly active fluidized-bed vinyl acetate catalyst on a support composed of inert microspheroidal particles composed of silicon oxide, zirconium oxide or aluminum oxide and having a defined pore distribution.

The article “Enhanced Voidage Correlations for Packed Beds of Various Particle Shapes and Sizes,” from Benyahia and O'Neill in Particulate Science and Technology 23:169-177 (2005) describes predictive mean voidage correlations for packed beds of spherical, cylindrical, and nonconventional cylindrical particles.

The article “Flow through Packed and Fluidized Beds,” from Subramanian (Clarkson University) describes pressure drop in packed and fluidized beds.

US 2021/0339229A1 discloses catalyst supports for use in packed bed reactors including ceramic material with geometric surface area per volume of ≥0.7 cm²/cm³ and a side crush strength of ≥250 kgf (or ≥1.5 cm²/cm³ and ≥150 kgf or ≥3 cm²/cm³ and ≥60 kgf) and a porosity of at least 6%. The catalyst supports are used in processes such as the steam reforming and production of direct-reduced iron.

The need remains for catalysts and catalyst shapes that lead to improved reactor pressure drop along with desirable selectivity, productivity, and space-time yield for the acetoxylation of olefins. These and other issues are disclosed in the present specification.

### BRIEF SUMMARY OF THE INVENTION

In some embodiments, the present disclosure is directed to a catalyst the acetoxylation of olefins comprising: a support material having a pimpled and/or dimpled external surface; and a catalytic layer disposed on the external surface, wherein the catalytic layer comprises palladium and gold, and wherein the catalyst comprises from 7 to 10 g/L palladium, from 3 to 8 g/L gold. The support material may be a pimpled sphere. The support material may have a diameter from 4 mm to 9 mm (e.g., from 4 mm to 8 mm or from 4 mm to 7 mm) as measured from the longest diameter of the sphere excluding the pimples. The diameter of the pimples, if the pimples were full spheres, may be from 0.5 mm to 8 mm or from 10% to 75% of the diameter of the sphere of the support material without the pimples. The pimples of the support material may have a length from their center, if the pimple were a full sphere, to the center of the sphere of the catalyst support material of from 0.1 mm to 5 mm.

In some embodiments, the support material comprises silica. The support material may also comprise alumina. In some embodiments, the support material comprises from 60 to 99 wt. % silica and from 1 to 40 wt. % alumina. In some embodiments, the catalyst may have a weight ratio of palladium to gold of ≤2:1, such as from 1.6:1 to 2:1.

In some embodiments, the catalyst may have a void fraction in a 250 mL graduated cylinder of 0.41 or greater. The catalyst layer may have a thickness from 100 to 300 μm. The catalytic layer may have an average penetration depth into the support material from 115 to 175 microns.

Some embodiments of the present invention involve a process for the acetoxylation of an olefin in a gaseous reaction stream containing an olefin, acetic acid, and an oxygen-containing gas, the process comprising passing a reaction gas over a reaction tube comprising a catalyst to form an acetoxylated olefin, wherein the catalyst comprises a support material having a pimpled and/or dimpled external surface; and a catalytic layer disposed on the external surface, wherein the catalytic layer comprises palladium and gold. In some embodiments, the olefin is ethylene and wherein the selectivity to vinyl acetate monomer is at least 60%. In some embodiments, the olefin is ethylene and wherein the conversion of ethylene is at least 15%. In some embodiments, the olefin is ethylene and the oxygen conversion is from 35 to 55%. In some embodiments, the olefin is ethylene and the selectivity to carbon dioxide is less than 10%. In some embodiments, the olefin is ethylene and the STY is at least 800 g vinyl acetate monomer/L/hour. The temperature across the reaction tube may range from 145 to 205° C. (e.g., 145 to 190° C.).

Additional embodiments and features are set forth in part in the description that follows, and in part will become apparent to those skilled in the art upon examination of the specification or may be learned by the practice of the invention. The features and advantages of the invention may be realized and attained by means of the instrumentalities, combinations, and methods described in the specification.

### BRIEF DESCRIPTION OF THE DRAWINGS

A further understanding of the nature and advantages of the present invention may be realized by reference to the remaining portions of the specification and the drawings wherein like reference numerals are used throughout the several drawings to refer to similar components. In some instances, a sublabel is associated with a reference numeral and follows a hyphen to denote one of multiple similar components. When reference is made to a reference numeral without specification to an existing sublabel, it is intended to refer to all such multiple similar components.

FIG. 1 is a schematic of the process used to form vinyl acetate monomer in accordance with embodiments of the present disclosure.

FIG. 2 is an illustration of two catalyst zones in accordance with embodiments of the present disclosure.

FIG. 3 is an illustration of three catalyst zones in accordance with embodiments of the present disclosure.

FIG. 4 is an illustration of a plurality of parallel reaction tubes in accordance with embodiments of the present disclosure.

FIG. 5 is an illustration of the surface area adjusted pressure drop (psi/2.84 ft² bed equivalent) versus the mass flux (lb/hr·ft²) for comparatives and example catalysts in accordance with the present disclosure.

### DETAILED DESCRIPTION OF THE INVENTION

### Introduction

As described herein, the present disclosure relates to catalysts, catalyst supports, catalyst shapes, and to processes for using the catalysts to acetoxylate an olefin. The catalyst may comprise a support material having an external surface. A catalytic layer may be disposed on the external surface of the support material. In some embodiments, the catalytic layer may be disposed on the support material in a thickness from 100 to 300 microns. In some embodiments, the support material (and the catalyst) can have a pimpled shape, for example a pimpled spherical shape. In other embodiments, the support material can have a dimpled shape, for example a dimpled spherical shape. In other embodiments, the support material can have both a pimpled and a dimpled shape.

The catalytic layer may be disposed on the support material by a variety of processes as described further herein. In some embodiments, the catalytic layer may comprise palladium and gold. The catalyst may comprise palladium in an amount from 7 to 10 g/L (1.1 to 1.6 wt. %) and gold in an amount from 3 to 8 g/L (0.47 to 1.3 wt. %), for example. In certain embodiments, the catalyst, including the support material and the catalytic layer, may have a diameter from greater than 1 millimeter (mm) to less than 10 mm, as measured from the longest diameter of the catalyst.

In some embodiments, the catalyst described herein may be used in a process to acetoxylate an olefin, e.g., to react ethylene with oxygen to form vinyl acetate monomer (VAM). The process may comprise providing a gaseous reaction stream containing an olefin, acetic acid, and an oxygen-containing gas, i.e., a reaction gas. This reaction gas may be passed over a reaction tube comprising the catalyst to form an acetoxylated olefin. The acetoxylated olefin may then be further processed.

Surprisingly and unexpectedly, the inventors found that when using the catalysts and/or catalyst supports described herein, various reactor efficiencies could be achieved, such as low pressure drop across the reaction tube and high catalyst surface area in an olefin acetoxylation reactor. Specifically, in certain embodiments, the catalyst support can be surface modified to increase the void volume. By increasing the void volume through surface modifications, the void fraction can be increased and the pressure drop of the gases flowing through the bed decreased. Modifications to the surface of spherical catalyst support pellets can also increase the surface area of the pellet, allowing either more catalytic surface area to be available, or the option of holding the total bed surface area constant and loading less catalyst for a “short load.” This second option could allow for a further decrease to pressure drop in the catalyst bed and a corresponding option to either increase mass flow through the reactor (at a constant power requirement in the compressor) or hold the mass flow constant and decrease the power requirements of the compressor.

As discussed further herein, the improved catalyst shapes and configurations allow for higher surface area and lower pressure drop across the reactor while achieving other objectives such as desirable catalyst life, high selectivity to VAM, low selectivity to carbon dioxide and heavy ends, and acceptable reactor temperature, space-time yield, and oxygen conversion. These and other details are described further herein.

### Acetoxylation of Olefins

As described herein, the catalyst may be used in a process for the acetoxylation of olefins. Exemplary olefins include ethylene and propylene, though others are also contemplated. The acetoxylation of ethylene to form vinyl acetate monomer is of particular industrial interest. The vinyl acetate monomer (VAM) is a compound represented by the following formula:

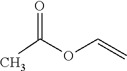

VAM is an important component in a wide variety of products, including polymers. VAM is also an important intermediate in coatings, textiles, paints, and other applications. For example, the polymer of VAM, polyvinyl acetate, is used in myriad applications including glues and adhesives.

The reaction to form VAM includes forming a gaseous reaction stream, e.g., a reaction gas. The reaction gas may comprise ethylene, acetic acid, and an oxygen-containing gas, e.g., molecular oxygen. In some aspects, the reaction gas may further comprise inert gases. The reaction gas may then be passed over a reaction tube comprising the catalyst.

In some aspects, the catalyst may be included in at least one fixed catalyst zone in the reaction tube and the reaction tube may comprise at least two fixed catalyst zones arranged in series. There may be multiple reaction tubes, arranged in parallel. Each reaction tube may comprise the same at least two fixed catalyst zones. The at least two fixed catalyst zones may comprise an inlet catalyst zone and an outlet catalyst zone. In the cases where more than two catalyst zones are present, the additional catalyst zones are understood to be between the inlet catalyst zone and outlet catalyst zone. Each catalyst zone comprises at least one catalyst.

The amount of catalyst loading in each catalyst zone may be selected depending on desired catalyst performance. In some aspects, the inlet catalyst zone may comprise from 5 to 70% of the catalyst loading of the entire reaction tube, e.g., of all catalyst in all catalyst zones in the reaction tube. In some aspects, the outlet catalyst zone may comprise from 30 to 95% of the catalyst loading of the entire reaction tube. In some aspects, a majority of the reaction tubes comprise approximately the same catalyst zones, allowing for slight differences based on aging the catalyst in each zone. The catalyst described herein may be used in the inlet catalyst zone, the outlet catalyst zone, or any additionally included catalyst zone.

The reaction may be conducted, in the gas phase, at a temperature from 100 to 250° C. and at a pressure from 1 to 25 bars, for example.

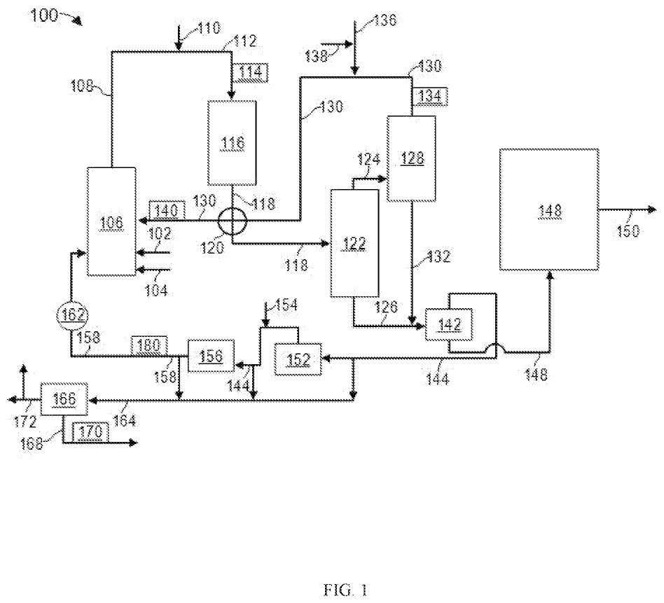

The acetoxylation of ethylene yields a crude vinyl acetate product comprising VAM water, and carbon dioxide as well as unreacted ethylene and acetic acid, which are used in excess. The ethylene and acetic acid are recycled back to the reactor from the reaction and purification sections of the unit. Product VAM is recovered and purified in the purification section and sent to storage tanks. Wastewater is sent to a treatment facility and carbon dioxide is vented to a pollution control device. Inert gases such as nitrogen and argon may accumulate over time and may then be purged from the reaction section to minimize buildup. A full description of the VAM production process is provided herein and illustrated in FIG. 1.

### VAM Production Process

FIG. 1 illustrates a process flow diagram of an example vinyl acetate production process 100 of the present disclosure. Additional components and modifications may be made to the process 100 without changing the scope of the present invention. Further, as would be recognized by one skilled in the art, the description of the process 100 and related system uses streams to describe the fluids passing through various lines. For each stream, the related system has corresponding lines (e.g., pipes or other pathways through which the corresponding fluids or other materials may pass readily) and optionally valves, pumps, compressors, heat exchangers, or other equipment to ensure proper operation of the system whether explicitly described or not.

Further, the descriptor used for individual streams does not limit the composition of said streams to consisting of said descriptor. For example, an ethylene stream does not necessarily consist of only ethylene. Rather, the ethylene stream may comprise ethylene and a diluent gas (e.g., an inert gas). Alternatively, the ethylene stream may consist of only ethylene. Alternatively, the ethylene stream may comprise ethylene, another reactant, and optionally an inert component.

In the illustrated process 100, an acetic acid stream 102 and an ethylene stream 104 are introduced to a vaporizer 106. Optionally, ethane may also be added to the vaporizer 106. In addition, one or more recycle streams 130, 158 (each described further herein) may also be introduced to the vaporizer 106. Optionally, one or more of the recycle streams 130, 158 may be combined with the acetic acid stream 102 (not shown) before introduction to the vaporizer 106.

The temperature and pressure of vaporizer 106 may vary over a wide range. The vaporizer 106 preferably operates at a temperature from 100° C. to 250° C., or from 100° C. to 200° C., or from 120° C. to 150° C. The operating pressure of the vaporizer 106 may range from 0.1 MPa to 2.03 MPa, or 0.25 MPa to 1.75 MPa, or 0.5 MPa to 1.5 MPa. The vaporizer 106 produces a vaporized feed stream 108. The vaporized feed stream 108 exits the vaporizer 106 and combines with an oxygen stream 110 to produce a combined feed stream 112. The combined feed stream 112 is analyzed by sensors 114 prior to being fed to a vinyl acetate reactor 116.

The sensors 114 include a water sensor for determining the concentration of water in the combined feed stream 112. The sensors 114 may optionally also include a temperature sensor, a pressure sensor, a flow rate sensor, composition sensors (e.g., gas chromatography, infrared spectroscopy, and oxygen analyzers) and the like, and any combination thereof. Each of the individual sensors may be present singularly or in a plurality. Having a plurality of a specific sensor provides redundancies that minimize downtime for sensor replacement and mitigate safety issues resulting from a failing or out of calibration sensor. While the sensors 114 are generally illustrated as upstream of the vinyl acetate reactor 116, said sensors may be placed in other locations where calculations may be performed to estimate a condition (e.g., temperature, pressure, or component concentration) at the reactor inlet or other suitable location.

The operating conditions in the vinyl acetate reactor 116 may be adjusted based on the composition of the combined feed stream 112. Generally, suitable ranges for the operating conditions in the vinyl acetate reactor 116 are provided below.

Regarding the general operating conditions of the vinyl acetate reactor 116, the molar ratio of ethylene to oxygen when producing vinyl acetate is preferably less than 20:1 in the vinyl acetate reactor 116 (e.g., 1:1 to 20:1, or 1:1 to 10:1, or 1.5:1 to 5:1, or 2:1 to 4:1). Further, the molar ratio of acetic acid to oxygen is preferably less than 10:1 in the vinyl acetate reactor 116 (e.g., 0.5:1 to 10:1, 0.5:1 to 5:1, or 0.5:1 to 3:1). The molar ratio of ethylene to acetic acid is preferably less than 10:1 in the vinyl acetate reactor 116 (e.g., 1:1 to 10:1, or 1:1 to 5:1, or 2:1 to 3:1). Accordingly, the combined feed stream 112 comprise the ethylene, oxygen, and acetic acid in said molar ratios.

The vinyl acetate reactor 116 may be a shell and tube reactor that is capable, through a heat exchange medium, of absorbing heat generated by the exothermic reaction and controlling the temperature therein within a temperature range of 100° C. to 250° C., or 110° C. to 200° C., or 120° C. to 180° C. The pressure in the vinyl acetate reactor 116 may be maintained at 0.5 MPa to 2.5 MPa, or 0.5 MPa to 2 MPa.

Further, the vinyl acetate reactor 116 may be a fixed bed reactor or a fluidized bed reactor, preferably a fixed bed reactor that containing reaction tubes as described herein.

Reactor 116 may comprise one or more tubes, e.g., a plurality of tubes arranged in parallel. In some aspects, the reactor may comprise from 2 to 10,000 tubes, e.g., from 50 to 10,000 tubes, from 500 to 10,000 tubes, from 1000 to 9500 tubes, or from 3000 to 9000 tubes. Each tube comprises the at least two fixed catalyst zones, described herein. In some aspects, at least 1% of the tubes comprise the same catalyst, e.g., at least 3%, at least 5%, at least 10%, e.g., at least 20%, at least 30%, at least 40%, at least 50%, at least 60%, at least 70%, at least 80%, at least 90%, at least 95%, at least 97.5%, at least 99%, at least 99.5%, or 100%, based on the total number of catalyst tubes. “The same catalyst” refers to the same catalyst size, shape, metal loading, and support, allowing for small differences due to manufacture.

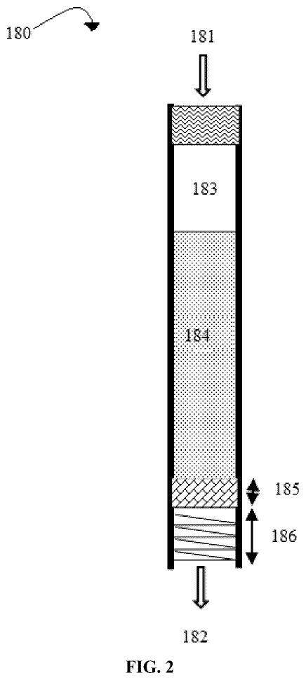

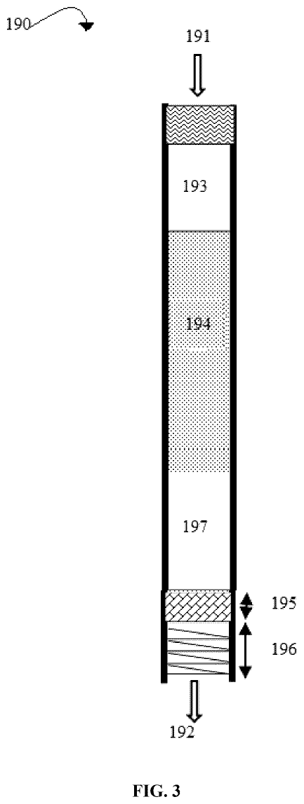

In some aspects, where the reaction tube comprises a zone loaded catalyst, at least 10% of the tubes comprise the same at least two fixed catalyst zones, e.g., at least 20%, at least 30%, at least 40%, at least 50%, at least 60%, at least 70%, at least 80%, at least 90%, at least 95%, at least 97.5%, at least 99%, at least 99.5%, or 100%, based on the total number of catalyst tubes. Two exemplary but non-limiting catalyst tube configurations as shown in FIG. 2 (tube 180) and FIG. 3 (tube 190). FIG. 2 shows a tube 180 showing inlet flow 181 into an open portion of the tube which may comprise inerts. These inerts may serve to filter the incoming flow to remove solids or liquids, similarly to a guard bed. Inlet catalyst zone 183 contains a fixed inlet catalyst. Outlet catalyst zone 184 contains a fixed outlet catalyst. The reaction gas then passes inerts 185, which may support the catalyst to prevent it from moving into/through the spring 186. The reaction gas then exits tube 180 in the direction of outlet flow 182. Similarly, FIG. 3 shows another non-limiting zone loaded catalyst configuration with tube 190 having an inlet flow 191, an inlet catalyst zone 193 comprising an inlet catalyst, an additional catalyst zone 194 comprising an additional catalyst, an outlet catalyst zone 197 comprising an outlet catalyst, inerts 195, spring 196, and outlet flow 192.

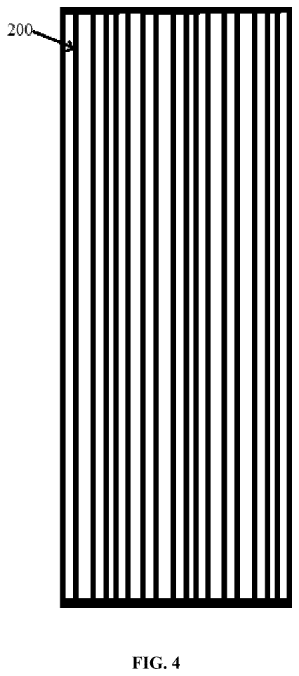

A multiple tube arrangement is shown in FIG. 4. FIG. 4 shows a plurality of tubes 200 in parallel but is not intended to be limiting in terms of the number of tubes. Tube 200 may be the tube 180, tube 190, or another tube configuration described herein.

Referring again to FIG. 1, because the process 100 uses one or more recycle streams 130, 156, 168 and diluents may be included, the components in the combined feed stream 112 are more than ethylene, acetic acid, and oxygen. Examples of components in the combined feed stream 112 where the concentration of said components may include, but are not limited to, ethylene, acetic acid, methane, ethane, propane, water, nitrogen, argon, and carbon dioxide.

Referring again to FIG. 1, the vinyl acetate reaction in the reactor 116 produces a crude vinyl acetate stream 118. Depending on conversion and reaction conditions, the crude vinyl acetate stream 118 may comprise 5 wt. % to 40 wt. % vinyl acetate, 5 wt. % to 40 wt. % acetic acid, 0.1 wt. % to 10 wt. % water, 10 wt. % to 80 wt. % ethylene, 1 wt. % to 40 wt. % carbon dioxide, 0.1 wt. % to 50 wt. % alkanes (e.g., methane, ethane, or mixtures thereof), and 0.1 wt. % to 15 wt. % oxygen. Optionally, the crude vinyl acetate stream 118 may also comprise 0.01 wt. % to 10 wt. % ethyl acetate. The crude vinyl acetate stream 118 may comprise other compounds such as methyl acetate, acetaldehyde, acrolein, propane, and inerts such as nitrogen or argon. Generally, these other compounds, except for inerts, are present in very low amounts.

The crude vinyl acetate stream 118 passes through a heat exchanger 120 to reduce the temperature of the crude vinyl acetate stream 118 and then to a separator 122 (e.g., a distillation column). Preferably, the crude vinyl acetate stream 118 is cooled to a temperature of 80° C. to 145° C., or 90° C. to 135° C., prior to being introduced into the separator 122. Preferably, no condensation of the liquefiable components occurs and the cooled crude vinyl acetate stream 118 is introduced to the separator 122 as gas.

The energy to separate the components of the crude vinyl acetate stream 118 may be provided by the heat of reaction in the reactor 116. In some embodiments, there may be an optional reboiler dedicated to increasing the separation energy within the separator 122. The separator 122 separates the crude vinyl acetate stream 118 into at least two streams: an overheads stream 124 and a bottoms stream 126. The overheads stream 124 may comprise ethylene, carbon dioxide, water, alkanes (e.g., methane, ethane, propane or mixtures thereof), oxygen, and vinyl acetate. The bottoms stream may comprise vinyl acetate, acetic acid, water, and potentially ethylene, carbon dioxide, and alkanes.

The overheads stream 124 is conveyed to a scrubber 128 to remove vinyl acetate in the overheads stream 124. As a result, the scrubber 128 has gas stream 130 and a bottoms stream 132. Vinyl acetate scrubbing may be achieved by passing the overheads stream 124 through a mixture of water and acetic acid.

The tail gas stream 130 comprises ethylene, carbon dioxide, alkanes, and oxygen. The conditions (e.g., temperature, pressure, and/or composition of components) of the tail gas stream 130 may be measured using sensors 134. The sensors 134 may include, but are not limited to, a temperature sensor, a pressure sensor, a flow rate sensor, composition sensors (e.g., gas chromatography, infrared spectroscopy, and oxygen analyzers) and the like, and any combination thereof. Each of the individual sensors may be present singularly or in a plurality. Having a plurality of a specific sensor provides redundancies that minimize downtime for sensor replacement and mitigate safety issues resulting from a failing or out of calibration sensor. While the sensors 134 are generally illustrated as downstream of the scrubber 128 along the tail gas stream 130, said sensors may be placed in other locations where calculations may be performed to estimate a condition (e.g., temperature, pressure, or component concentration) of the tail gas stream 130 post scrubber 128.

The tail gas stream 130 (also referred to as a recycle stream) is conveyed back to the vaporizer 106 through the heat exchanger 120, where the crude vinyl acetate stream 118 heats the tail gas stream 130. Optionally, the tail gas stream 130 may be augmented with or otherwise have added thereto other streams including other recycle streams (not shown) in the process and feed streams. As illustrated, an ethylene feed steam 136 and a methane feed stream 138 (or other ballast gas stream) are combined (e.g., mixed with or entrained with) with the tail gas stream 130.

Additionally, between the scrubber 128 and the heat exchanger 120, other processes (not illustrated) may be performed on the tail gas stream 130. For example, the tail gas stream 130 may have at least a portion of the carbon dioxide removed.

Between the heat exchanger 120 and the vaporizer 106, the tail gas stream 130 is analyzed by sensors 140. Examples of sensors 140 include, but are not limited to, a temperature sensor, a pressure sensor, a flow rate sensor, composition sensors (e.g., gas chromatography, infrared spectroscopy, and oxygen analyzers) and the like, and any combination thereof. Each of the individual sensors may be present singularly or in a plurality. Having a plurality of a specific sensor provides redundancies that minimize downtime for sensor replacement and mitigate safety issues resulting from a failing or out of calibration sensor. While the sensors 140 are generally illustrated as between the heat exchanger 120 and the vaporizer 106, said sensors may be placed in other locations where calculations may be performed to estimate a condition (e.g., temperature, pressure, or component concentration) between the heat exchanger 120 and the vaporizer 106 or other suitable location.

Referring again to FIG. 1, the bottoms stream 126 from the separator 122 and the bottoms stream 132 from the scrubber 128 may be combined and fed to a crude tank 142. Generally, the stream(s) coming into the crude tank 142 are depressurized to a pressure of 0.1 MPa to 0.15 MPa. In depressurizing the incoming stream, the ethylene, carbon dioxide, inert gases (e.g., nitrogen and/or argon), and acetic acid flash to produce a flash gas stream 144. The bottoms of the crude tank 142 primarily comprise vinyl acetate, water, and acetic acid with some ethyl acetate byproduct. The bottoms are transported as a vinyl acetate stream 146 to be purified by various processes 148 to produce the purified vinyl acetate product stream 150. Examples of purification processes 148 include, but are not limited to, azeotrope distillation, water stripping, distillation, phase separations, and the like, and any combination thereof. Examples of different processing methods and systems are described in U.S. Pat. Nos. 6,410,817, 8,993,796, and 9,045,413 and US Patent App. Pub. No. 2014/0066649, each of which is incorporated herein by reference.

Further, the purification processes 148 may produce additional streams that individually or in any combination may be recycled back to the vaporizer 106, the tail gas stream 130, the flash gas stream 144, and/or other streams within the process 100.

Optionally (not shown), a portion of the tail gas slip stream 130 may be combined with (e.g., mixed with or entrained with) the flash gas stream 144.

At least a portion of the carbon dioxide in the flash gas stream 144 (optionally having been combined with a portion of the tail gas slip stream 130) is removed before recycling back into the vaporizer 106. As illustrated, the flash gas stream 144 first passes through a CO₂ scrubber 152 and then a CO₂ absorber 156 to produce a CO₂ removal overheads stream 158. Between the CO₂ scrubber 152 and the CO₂ absorber 156, ethylene may be added to the flash gas stream 144 from ethylene stream 154.

After the CO₂ absorber 156, the CO₂ removal overheads stream 158 is analyzed by sensors 160. Examples of sensors 160 include, but are not limited to, a temperature sensor, a pressure sensor, a flow rate sensor, composition sensors (e.g., gas chromatography, infrared spectroscopy, and oxygen analyzers) and the like, and any combination thereof. Each of the individual sensors may be present singularly or in a plurality. Having a plurality of a specific sensor provides redundancies that minimize downtime for sensor replacement and mitigate safety issues resulting from a failing or out of calibration sensor. While the sensors 160 are generally illustrated as downstream of the CO₂ absorber 156, said sensors 160 may be placed in other locations where calculations may be performed to estimate a condition (e.g., temperature, pressure, or component concentration) downstream of the CO₂ absorber 156 or other suitable location.

Referring again to FIG. 1, the CO₂ removal overheads stream 158 may then be passed through a heat exchanger 162 and fed into the vaporizer 106. Further, a slip stream 164 from the flash gas stream 144 and/or the CO₂ removal overheads stream 158 is used to purge inerts from the system. This slip stream 164 may be sent through an ethylene recovery process 166. The ethylene recovery process 162 produces an ethylene vent stream 168 and a recycle stream 172. Examples of ethylene recovery processes 166 may include, but are not limited to, scrubbing systems, membrane recovery processes, and the like, and any combination thereof. The ethylene recovery processes 166 may produce a vent stream 168 and additional stream(s) 172 that take the ethylene recovered to other processes or for recycling back into this process 100.

Accordingly, after the ethylene recovery process 166, the ethylene vent stream 168 is analyzed by sensors 170. Examples of sensors 170 include, but are not limited to, a temperature sensor, a pressure sensor, a flow rate sensor, composition sensors (e.g., gas chromatography, infrared spectroscopy, and oxygen analyzers) and the like, and any combination thereof. Each of the individual sensors may be present singularly or in a plurality. Having a plurality of a specific sensor provides redundancies that minimize downtime for sensor replacement and mitigate safety issues resulting from a failing or out of calibration sensor. While the sensors 170 are generally illustrated along the ethylene vent stream 168, said sensors 170 may be placed in other locations where calculations may be performed to estimate a condition (e.g., temperature, pressure, or component concentration) ethylene vent stream 168 or other suitable location.

### The Catalyst

As described herein, the catalyst may comprise a support material having an external surface and a catalytic layer disposed on the external surface. The support material may be referred to as a refractory support. The refractory support may include a metal oxide such as silica, silica-alumina, titania, or zirconia. In some aspects, the refractory support is silica. In some further aspects, the refractory support is silica-alumina. In these aspects, the support may comprise from 60 to 99 wt. % silica and from 1 to 40 wt. % alumina, based on the entire weight of the support (e.g., from 1 to 30 wt. % alumina, from 1 to 20 wt. % alumina, from 1 to 10 wt. % alumina, from 1 to 5 wt. % alumina, from 2 to 5 wt. % alumina, from 3 to 5 wt. % alumina, or from 3 to 4.5 wt. % alumina.

In some embodiments, the support material, and thus the catalyst, may have a pimpled cross-sectional shape including, but not limited to, pimpled cylindrical, pimpled tubular, pimpled polylobular, pimpled ring, pimpled star, pimpled trilobe, pimpled quadrilobe, pimpled cloverleaf shape, pimpled saddle, pimpled fluted, pimpled ridged, pimpled multi-pointed star, pimpled hollow cylinder, pimpled cogwheel, pimpled spoked wheel, pimpled multi-hole pellet, pimpled monolith, pimpled T-fin shape, pimpled Raschig ring shape, pimpled spherical shape, or other pimpled shapes. In some aspects, the shape is pimpled and spherical, where the sphere itself (aside from the pimples) deviates from a perfect sphere in an amount that is sufficiently small so as to not measurably detract from spherical shape. The exact degree of deviation allowable may in some cases depend on the specific context. In some aspects, the support material and thus the catalyst are in a pimpled Raschig ring shape. In some aspects, the support material and thus the catalyst are in a pimpled T-fin shape having a plurality of T-fin extensions. The cross-sectional shape of the support material may be selected depending on the desired surface area since shapes like a T-fin may allow for more surface area of the support to be coated with the catalytic layer.

In some embodiments, the support material, and thus the catalyst, may have a dimpled cross-sectional shape including, but not limited to, dimpled cylindrical, dimpled tubular, dimpled polylobular, dimpled ring, dimpled star, dimpled trilobe, dimpled quadrilobe, dimpled cloverleaf shape, dimpled saddle, dimpled fluted, dimpled ridged, dimpled multi-pointed star, dimpled hollow cylinder, dimpled cogwheel, dimpled spoked wheel, dimpled multi-hole pellet, dimpled monolith, dimpled T-fin shape, dimpled Raschig ring shape, dimpled spherical shape, or other dimpled shapes. In some aspects, the shape is dimpled and spherical, where the sphere itself (aside from the dimples) deviates from a perfect sphere in an amount that is sufficiently small so as to not measurably detract from spherical shape. The exact degree of deviation allowable may in some cases depend on the specific context. In some aspects, the support material and thus the catalyst are in a dimpled Raschig ring shape. In some aspects, the support material and thus the catalyst are in a dimpled T-fin shape having a plurality of T-fin extensions.

In some embodiments involving pimples, the pimples can have as shape that is some portion of a sphere. Thus, in some embodiments, individual pimples on the support material (and the catalyst) can have a diameter of from 0.1 mm to 10 mm (e.g., from 0.2 mm to 10 mm, from 0.5 mm to 8 mm, from 1 mm to 8 mm, from 1 mm to 5 mm, from 1.5 mm to 4 mm, or from 2 mm to 3 mm) if the pimples were full spheres. In other words, the pimples have some portion of the curvature of a sphere with the aforementioned diameters. In some embodiments, all the pimples on a single catalyst support have substantially the same diameter (if the pimples were full spheres). In other embodiments, the pimples on a single catalyst support can have varying diameters (if the pimples were full spheres). In some embodiments, the diameter of the individual pimples (if the pimples were full spheres) can be from 5% to 80% of the diameter of the main body of the catalyst support (i.e., the catalyst support without the pimples) (e.g., from 10% to 75%, from 15% to 70%, from 20% to 60%, from 25% to 60%, or from 30% to 50%).

In some embodiments where the pimples are some portion of a sphere, the pimples can be from 1% to 70% of a full sphere (e.g., from 2% to 50% of a sphere, from 5% to 40% of a sphere, from 8% to 45% of a sphere, from 10% to 40% of a sphere, or from 15% to 35% of a sphere), based on volume.

In some embodiments, individual pimples on the support material (and the catalyst) can have a length from the outer edge of the pimple to the outer edge of the main body of the catalyst support (i.e., the catalyst support absent the pimples) along the radius of the pimple (if the pimple were a full sphere) of from 0.01 mm to 5 mm (e.g., from 0.05 mm to 5 mm, from 0.1 mm to 5 mm, from 0.2 mm to 4 mm, from 0.5 mm to 4 mm, from 1 mm to 4 mm, from 1.5 mm to 3 mm, or from 1.75 mm to 3 mm). In some embodiments, the pimples on a single catalyst support have the same length from the outer edge of the pimple to the outer edge of the main body of the catalyst support along the radius of the pimple (if the pimple were a full sphere). In other embodiments, the pimples on a single catalyst support can have different lengths from the outer edge of the pimple to the outer edge of the main body of the support along the radius of the pimple (if the pimple were a full sphere). In some embodiments, individual pimples on the catalyst support (and the catalyst) can have a length from the outer edge of the pimple to the outer edge of the main body of the support along the radius of the pimple (if the pimple were a full sphere) that is from 1% to 70% of the diameter of the main body of the catalyst support (e.g., from 5% to 75%, from 10% to 70%, from 15% to 60%, from 20% to 60%, or from 25% to 50%). These lengths discussed can be utilized with any pimple diameters (if the pimple were a full sphere), for instance any of those discussed above.

In some embodiments, individual pimples on the catalyst support material (and the catalyst) can have a length from the center of the pimple (if the pimple were a full sphere) to the center of the main body of the catalyst support along the extended radius of the pimple of from 0.01 mm to 5 mm (e.g., from 0.05 mm to 5 mm, from 0.1 mm to 5 mm, from 0.2 mm to 4 mm, from 0.5 mm to 4 mm, from 1 mm to 4 mm, from 1.5 mm to 3 mm, or from 1.75 mm to 3 mm). In some embodiments, the pimples on a single catalyst support have the same length from the center of the pimple (if the pimple were a full sphere) to the center of the main body of the catalyst support along the extended radius of the pimple. In other embodiments, the pimples on a single catalyst support can have different lengths from the center of the pimple (if the pimple were a full sphere) to the center of the main body of the catalyst support along the extended radius of the pimple. In some embodiments, individual pimples on the catalyst support material (and the catalyst) can have a length from the center of the pimple (if the pimple were a full sphere) to the center of the main body of the catalyst support along the extended radius of the pimple that is from 1% to 90% of the diameter of the main body of the catalyst support (e.g., from 5% to 80%, from 10% to 75%, from 15% to 70%, from 20% to 60%, or from 25% to 50%). These lengths discussed can be utilized with any pimple diameters (if the pimple were a full sphere), for instance any of those discussed above.

In some embodiments, the pimples can be positioned from 5 degree to 90 degree rotations along the x, y, and z, axis of the main body of the catalyst support material (e.g, from 10 degree to 80 degree, 15 degree to 75 degree, 20 degree to 70 degree, 25 degree to 65 degree, 30 degree to 60 degree, 35 degree to 55 degree, 40 degree to 50 degree, or about 45 degrees).

In some embodiments, an individual catalyst support (and thus, the catalyst) can contain from 2 to 60 pimples (i.e., on each individual catalyst support material or catalyst) (e.g., from 3 to 50 pimples, from 4 to 45 pimples, from 5 to 50 pimples, from 5 to 40 pimples, from 6 to 50 pimples, from 7 to 40 pimples, from 8 to 40 pimples, from 10 to 35 pimples, from 12 to 30, pimples, or from 15 to 25 pimples).

In some embodiments regarding dimples, the void of the dimples can have a shape that is some portion of a sphere. Thus, in some embodiments, individual dimples on the support material (and the catalyst) can have a diameter of from 0.1 mm to 10 mm (e.g., from 0.2 mm to 10 mm, from 0.5 mm to 8 mm, from 1 mm to 8 mm, from 1 mm to 5 mm, from 1.5 mm to 4 mm, or from 2 mm to 3 mm) if the dimples (i.e., the indented void) were full spheres. In other words, the voids of the dimples have some portion of the curvature of a sphere with the aforementioned diameters. In some embodiments, the dimples on a single support material have the same diameter (if the dimples were full spheres). In other embodiments, the dimples on a single support material can have varying diameters (if the dimples were full spheres). In some embodiments, the diameter of the individual dimples (if the dimples were full spheres) can be from 5% to 80% of the diameter of the catalyst support material (e.g., from 10% to 75%, from 15% to 70%, from 20% to 60%, from 25% to 60%, or from 30% to 50%).

In some embodiments where the dimple voids are some portion of a sphere, the dimple voids can be from 1% to 70% of a full sphere (e.g., from 2% to 50% of a sphere, from 5% to 40% of a sphere, from 8% to 45% of a sphere, from 10% to 40% of a sphere, or from 15% to 35% of a sphere), based on volume.

In some embodiments, individual dimples on the support material (and the catalyst) can have a length from the deepest inner edge of the dimple to the extended edge of the catalyst support material along the radius of the dimple (if the void of the dimple were a full sphere) of 0.01 mm to 5 mm (e.g., from 0.05 mm to 5 mm, from 0.1 mm to 5 mm, from 0.2 mm to 4 mm, from 0.5 mm to 4 mm, from 1 mm to 4 mm, from 1.5 mm to 3 mm, or from 1.75 mm to 3 mm). In some embodiments, the dimples on a single support material have the same length from the outer edge of the dimple to the outer edge of the main body of the catalyst support material along the radius of the dimple (if the void of the dimple were a full sphere). In other embodiments, the dimples on a single support material can have different lengths from the outer edge of the dimple to the outer edge of the main body of the support material along the radius of the dimple (if the void of the dimple were a full sphere). In some embodiments, individual dimples on the support material (and the catalyst) can have a length from the outer edge of the dimple to the outer edge of the main body of the support material along the radius of the dimple (if the void of the dimple were a full sphere) that is from 1% to 70% of the diameter of the main body of the catalyst support material (e.g., from 5% to 75%, from 10% to 70%, from 15% to 60%, from 20% to 60%, or from 25% to 50%). These lengths discussed can be utilized with any dimple diameters (if the void of the dimple were a full sphere), for instance any of those discussed above.

In some embodiments, individual dimples on the support material (and the catalyst) can have a length from the center of the dimple (if the void of the dimple were a full sphere) to the center of the main body of the catalyst support material along the extended radius of the dimple of from 0.01 mm to 5 mm (e.g., from 0.05 mm to 5 mm, from 0.1 mm to 5 mm, from 0.2 mm to 4 mm, from 0.5 mm to 4 mm, from 1 mm to 4 mm, from 1.5 mm to 3 mm, or from 1.75 mm to 3 mm). In some embodiments, the dimples on a single support material have the same length from the center of the dimple (if the void of the dimple were a full sphere) to the center of the main body of the catalyst support material along the extended radius of the dimple. In other embodiments, the dimples on a single support material can have different lengths from the center of the dimple (if the void of the dimple were a full sphere) to the center of the main body of the catalyst support material along the extended radius of the dimple. In some embodiments, individual dimples on the support material (and the catalyst) can have a length from the center of the dimple (if the void of the dimple were a full sphere) to the center of the main body of the catalyst support material along the extended radius of the dimple that is from 1% to 90% of the diameter of the main body of the catalyst support material (e.g., from 5% to 80%, from 10% to 75%, from 15% to 70%, from 20% to 60%, or from 25% to 50%). These lengths discussed can be utilized with any dimple diameters (if the void of the dimple were a full sphere), for instance any of those discussed above.

In some embodiments, the dimples can be positioned from 5 degree to 90 degree rotations along the x, y, and z, axis of the main body of the catalyst support material (e.g, from 10 degree to 80 degree, 15 degree to 75 degree, 20 degree to 70 degree, 25 degree to 65 degree, 30 degree to 60 degree, 35 degree to 55 degree, 40 degree to 50 degree, or about 45 degrees).

In some embodiments, an individual catalyst support (and thus, the catalyst) can contain from 2 to 60 dimples (i.e., on each individual catalyst support material or catalyst) (e.g., from 3 to 50 dimples, from 4 to 45 dimples, from 5 to 50 dimples, from 5 to 40 dimples, from 6 to 50 dimples, from 7 to 40 dimples, from 8 to 40 dimples, from 10 to 35 dimples, from 12 to 30, dimples, or from 15 to 25 dimples).

In some embodiments, the catalyst support shapes can be prepared using 3-D printing techniques.

The catalytic layer may be disposed on the external surface of the support material. Various methods may be used. For example, in some aspects, the catalytic layer may be disposed on the support material by spray drying. US Pub. 2015/0126361, incorporated by reference herein, describes an exemplary spray drying method. Generally, the method includes: (a) introducing a support body into a coating device; (b) applying a Pd precursor compound and an Au precursor compound, in each case in dissolved form, to the support body by spray coating in the coating device; (c) drying the support body coated with the precursor compounds in the coating device; (d) reducing the metal components of the precursor compounds to the elemental metals in the coating device; and (e) removing the support body from the coating device. More specifically, the spray drying method includes spraying metal precursor compounds through a spray novel into an apparatus into which a spray gas is fed. The gas may have a non-reductive action, such as air or an inert gas, and is fed at a pressure from 1 to 1.8 bar, e.g., from 1 to 1.6 or 1.1 to 1.4 bar. The spraying rate and pressure may be chosen for the nozzle used and for a droplet size of a resultant aerosol from 1 to 100 microns, e.g., from 10 to 40 microns. The spray nozzle may be an IRN010 PEEK-type Rotojet spray nozzle from Innojet.

Alternative methods for applying the catalytic layer may be used, including impregnation, as described in US Pub. No. 2010/0190638, incorporated by reference herein. In such instances, a mixed solution of a Pd-containing precursor compound and an Au-containing precursor compound is normally applied to support bodies in a coating device. Then, the support body is dried in a drying device The metal components of the precursor compounds are then converted to the elemental metals in a reduction furnace. Then, the usually wet-chemical impregnation of the reduced support bodies with potassium acetate takes place.

In some embodiments, the catalytic layer may be applied in a thickness from 10 to 300 microns. The thickness of the layer is substantially homogenous across the external surface of the support material, e.g., varies in thickness by less than 10 microns. In some aspects, the thickness of the coating on the refractory support may range from 110 to 300 microns, e.g., from 115 to 200 microns, from 120 to 175 microns, from 125 to 150 microns, from 200 to 300 microns, or from 225 to 275 microns. In some aspects, the thickness of the coating may be selected based on the size of the catalyst. The size of the catalyst, as measured by the largest diameter of the main body of the catalyst (i.e., without dimples or pimples), may range from 1 mm to 10 mm, e.g., from 4 mm to 8 mm, from 5 mm to 7 mm, 5.1 to 6.9 mm, from 5.2 to 6.8 mm, from 5.3 to 6.7 mm, from 5.4 to 6.6 mm, or from 5.5 to 6.5 mm. In some aspects, the catalyst diameter is approximately 5 mm. In some aspects, the catalyst diameter is approximately 6 mm. In some aspects, the catalyst diameter is approximately 7 mm. The size of the catalyst may be based on average measurements of the catalyst, e.g., a plurality of catalyst. In some embodiments, the average size of the catalyst may vary by less than 0.1 mm. The catalyst diameter may be measured according to ASTM UOP947-96 (1996).

In some embodiments, the catalytic layer of the catalyst comprises palladium (Pd) and gold (Au). The catalyst (total weight, i.e., including the support material and catalytic layer) may comprise from 7 to 10 g/L Pd, e.g., from 7 to 9.5 g/L, from 7 to 9 g/L, from 7.2 to 8.8 g/L, from 7.2 to 8.6 g/L, from 7.6 to 8.4 g/L, from 7.9 to 8.4 g/L, from 8.0 to 8.4 g/L, from 8.1 to 8.4 g/L, from 8.2 to 8.4 g/L, from 7.9 to 8.3 g/L, from 7.9 to 8.2 g/L, or from 7.9 to 8.1 g/L. In terms of weight percent, the catalyst may comprise less than 1.6 wt. % Pd, e.g., less than 1.5 wt. %, less than 1.4 wt. %, or less than 1.3 wt. %. In terms of ranges, the catalyst may comprise from 1.1 to 1.6 wt. % Pd, e.g., from 1.15 to 1.5 wt. %, from 1.2 to 1.4 wt. %, or from 1.2 to 1.3 wt. %.

The catalyst may comprise gold in an amount from 3 to 8 g/L Au, e.g., from 3.5 to 7 g/L, from 3.5 to 6 g/L, from 3.5 to 5 g/L, from 3.8 to 4.5 g/L, from 3.8 to 4.2 g/L, or from 3.8 to 4.1 g/L. In terms of weight percent, the catalyst may comprise less than 1.3 wt. % Au, e.g., less than 1.2 wt. %, less than 1.1 wt. %, or less than 1 wt. %. In terms of ranges, the catalyst may comprise from 0.4 to 1.3 wt. % Au, e.g., from 0.5 to 1.2 wt. %, from 0.5 to 1.1 wt. %, from 0.6 to 1 wt. %, from 0.6 to 0.8 wt. %, from 0.6 to 0.75 wt. %, from 0.6 to 0.7 wt. %, from 0.65 to 0.75 wt. %, or from 0.65 to 0.70 wt. %.

The catalytic layer of the catalyst may also comprise potassium acetate (KOAc). The KOAc may be present in the greatest amount, as compared to Pd and Au. The KOAc may be present, in the catalyst, from 25 to 55 g/L, e.g., from 30 to 50 g/L, from 35 to 45 g/L, from 37.5 to 42.5 g/L, or approximately 40 g/L. In terms of weight percent, the KOAc may be present from 3.0 to 10.0 wt. %, from 4.0 to 9.0 wt. %, from 4.5 to 8.5 wt. %, from 5.0 to 7.5 wt. %, or from 5.5 to 7.0 wt. %.

In some aspects, the ratio of Pd to Au may be from 1:2 to 3.5:1 Pd to Au on a g/L basis. In some aspects, the ratio of Pd to Au may be from <2.0:1 on a g/L basis. For example, the ratio of Pd to Au may range from 1:2 to 3.5:1, on g/L basis, e.g., from 1:1 to 3.5:1, from 1:1 to 3:1, from 1:1 to 2:1, from 1.6:1 to 2:1, from 1.6:1 to 1.9:1, from 1.6:1 to 1.8:1, from 1.6:1 to 1.7:1, from 1.7:1 to 2:1, or from 1.8:1 to 1.9:1 on a g/L basis.

For purposes of calculating the weight percent of components on the catalyst from the g/L measurement, a density of 630 g/L may be used. Additionally, the g/L and weight percentages are measured based on the total weight of the catalyst, including the catalytic layer and the support material. The support material may make up the remaining amount of catalyst after Pd, Au, KOAc, and any other components are included.

In some embodiments, an individual catalyst particle can have a weight from 0.01 g to 1.0 g (e.g., from 0.02 g to 1.0 g, from 0.05 g to 0.8 g, from 0.07 g to 0.7 g, from 0.08 g to 0.5 g, from 0.1 g to 0.5 g, or from 0.1 g to 0.4 g).

In some embodiments, the catalyst can have a void fraction in a 250 mL graduated cylinder of from 0.2 to 0.8 (e.g., from 0.3 to 0.6, from 0.35 to 0.5, from 0.4 to 0.45, or from 0.41 to 0.44). In some embodiments, the catalyst can have a void fraction in a 250 mL graduated cylinder of 0.41 or greater.

In some embodiments, the catalyst can have a surface area per particle of from 0.1 mm² to 500 mm² (e.g., from 1 mm² to 500 mm², from 5 mm² to 300 mm², from 10 mm² to 200 mm², from 10 mm² to 150 mm², from 20 mm² to 100 mm², from 25 mm² to 100 mm², from 50 mm² to 100 mm², from 60 mm² to 90 mm², or from 70 mm² to 90 mm²).

In some aspects, the average penetration depth of the catalytic layer into the support material, as measured by EDS line scans, ranges from 115 to 175 microns, e.g., from 120 to 170 microns, from 125 to 165 microns, from 130 to 160 microns, or from 135 to 155 microns. The average penetration depth of the catalytic layer into the support material may influence the activity of the catalyst, including both the selectivity to product, e.g., VAM, and the selectivity to carbon dioxide. By having an average penetration depth from 115 to 175 microns, the catalyst described herein may be used to balance high catalyst activity with low carbon dioxide selectivity, thus increasing the productivity of the overall acetoxylation process.

Activity and selectivity of the catalysts in the following examples and comparative examples are measured over a time of up to 200 hours. The catalysts are tested in a flow tube whose temperature is controlled by means of oil (reactor length 1200 mm, internal diameter 19 mm) at an absolute pressure of 9.8 bar and a space velocity (GHSV) of 4000-8000 standard m³/(m³*h) using the following gas composition: 60% by volume of ethylene, 19.5% by volume of argon, 13% by volume of acetic acid and 7.5% by volume of oxygen. The catalyst systems are tested in the temperature range from 130 to 180° C. (gas entry temperature upstream of the catalyst bed). To characterize the course of the reaction, the temperature profile is measured by means of a multi-point temperature sensor in the catalyst bed. The reaction products and unreacted starting materials are analyzed at the output of the reactor by means of on-line gas chromatography. The space-time yield of the catalyst system in gram of vinyl acetate monomer per hour and liter of catalyst (g (VAM)/l of cat.*h) is determined as a measure of the catalyst activity. The selectivity is determined via the ratio of vinyl acetate formed to ethylene reacted.

In addition to determination of the reaction products in the gas phase, the liquid reaction products are condensed in a vessel maintained at from 10 to 15° C. and the condensate obtained is analyzed by means of gas chromatography.

The acetoxylation of olefins, e.g., ethylene, may achieve favorable conversion of ethylene and favorable selectivity and productivity to vinyl acetate monomer. For purposes of the present invention, the term “conversion” refers to the amount of ethylene in the feed that is converted to a compound other than ethylene. Conversion is expressed as a percentage based on ethylene in the feed. The conversion may be at least 30%, e.g., at least 40%, or at least 60%. Although catalysts that have high conversions are desirable, such as at least 60%, in some embodiments a low conversion may be acceptable at high selectivity for VAM. It is, of course, well understood that in many cases, it is possible to compensate for conversion by appropriate recycle streams or use of larger reactors/more reactor tubes, but it is more difficult to compensate for poor selectivity.

Selectivity is expressed as a mole percent based on converted ethylene. It should be understood that each compound converted from ethylene has an independent selectivity and that selectivity is independent from conversion. For example, if 60 mole % of the converted ethylene is converted to vinyl acetate monomer, the vinyl acetate monomer selectivity is 60%. In one embodiment, catalyst selectivity to vinyl acetate monomer is at least 60%, e.g., at least 70%, at least 80%, or at least 90%. Preferably, the selectivity to vinyl acetate monomer is at least 80%, e.g., at least 85% or at least 88%. Preferred embodiments of the process also have low selectivity to undesirable products, such as heavy ends and carbon dioxide. The selectivity to these undesirable products preferably is less than 10%, e.g., less than 5% or less than 2%.

The selectivity and conversion relating to the reaction are functions of several variables including reactor temperature, component concentration, and the condition of the catalyst. Deactivation of the catalyst, which routinely occurs over time due to buildup of tars and polymeric materials on the catalyst surface and/or to structural changes of the catalyst metals, may adversely affect the reaction process, particularly with regard to selectivity. These changes in reactor performance may ultimately lead to compositional changes in the liquid stream entering the purification section of a vinyl acetate plant.

Generally, the performance of the zone loading may be reviewed by at least one of three measures: oxygen conversion, space-time yield, and the temperature through the reactor In some aspects, two of these measures are used.

Oxygen conversion refers to the amount of oxygen provided to the reaction tube that is converted, similar to the measure of ethylene conversion. In some aspects, the oxygen conversion ranges from 35 to SS %, e.g., from 40 to 55%, or from 45 to 50%.

Space-time yield refers to the amount of product made per packed volume bed per unit time. In some aspects, the space-time yield (STY) is at least 800 g VAM/L/hour and may range from 800-1600 g VAM/L/hour, e.g., from 900 to 1500 g VAM/L/hour, or from 1000 to 1500 g VAM/L/hour.

The temperature across the reaction tube may range from 145 to 205° C., e g. 145 to 190° C., from 150 to 180° C., from 155 to 175° C., or from 160 to 170° C. Without being bound by theory, it is believed that the temperature may vary based on the catalyst age, with a newer catalyst typically tracking with a lower temperature than an older catalyst. A lower temperature across the reaction tube may indicate slower deactivation of the catalyst as compared to a higher temperature. A lower temperature may also indicate lower carbon dioxide selectivity which corresponds to more product made Since ethylene is a relatively expensive component, operating at a lower temperature may burn less ethylene and may improve process efficiency. The temperature may be balanced with conversion, selectivity, and costs in replacing catalysts.

In some aspects, using the catalyst disclosed herein may advantageously achieve a desirable pressure drop across the reaction tube. In some aspects, the pressure drop may range from 12 to 30 psi, e.g., from 15 to 25 psi, or from 17.5 to 22.5 psi. The pressure drop may be measured by measuring the absolute pressure at the inlet of the reactor and the outlet of the reactor and then calculate the difference.

### Zone Loaded Catalyst(s)

As described herein, in some aspects, the reaction tube(s) include zone loaded catalysts comprising the catalyst described herein in at least the inlet catalyst zone, the outlet catalyst zone, or any additional catalyst zone.

As illustrated in FIG. 2, tube 180 comprises an inlet catalyst zone 183 and an outlet catalyze zone 184. The inlet catalyst zone may comprise one or more catalysts, referred to as inlet catalysts. Similarly, the outlet catalyst zone may comprise one or more catalysts, referred to as outlet catalysts. It is also contemplated, as shown in FIG. 3, that one or more catalyst zones may be included between the inlet catalyst zone and outlet catalyst zone, referred to as additional catalyst zone(s), e.g., first additional catalyst zone, second additional catalyst zone, etc. Each catalyst zone may contain a single catalyst.

In some aspects, the inlet catalyst zone comprises from 5 to 70% of the catalyst loading, based on 100% by weight of the catalyst loading of all catalyst zones, e.g., from 10 to 70%, from 15 to 70%, from 20 to 70%, from 25 to 70%, from 30 to 70%, from 35 to 70%, from 40 to 70%, from 45 to 70%, from 50 to 70%, from 50 to 65%, from 15 to 65%, from 15 to 60%, from 15 to 55%, from 20 to 50%, from 20 to 40%, from 20 to 35%, or from 20 to 30%.

In some aspects, the outlet catalyst zone comprises from 30 to 95% of the catalyst loading, based on 100% by weight of the catalyst loading of all catalyst zones, e.g., from 35 to 90%, from 35 to 80%, from 40 to 80%, from 40 to 75%, from 40 to 70%, from 45 to 70%, from 50 to 70%, from 55 to 70%, from 55 to 95%, from 55 to 90%, from 55 to 85%, from 55 to 80%, from 60 to 80%, from 65 to 80%, or from 70 to 80%.

In some aspects, at least one additional catalyst zone may be present. In such aspects, the at least one additional catalyst zone may comprise from 1 to 25% of the catalyst loading, based on the catalyst loading of all catalyst zones, e.g., from 5 to 20%, or from 10 to 15%.

In some aspects, the inlet catalyst zone comprises more of the catalyst loading than the outlet catalyst zone, e.g., greater than 50% by weight based on the weight of all catalyst loading in all catalyst zones. In other aspects, the inlet catalyst zone comprises less of the catalyst loading than the outlet catalyst zone, e.g., less than 50% by weight based on the weight of all catalyst loading in all catalyst zones.

In some aspects, in addition to the catalyst described herein, different catalyst(s), referred to as zone loading catalyst(s) may be used. The zone loading catalyst(s) may be included as the inlet, outlet, and/or additional catalyst and may comprise palladium (Pd). In some aspects, the inlet catalyst comprises from 4 to 15 g/L Pd, e.g., from 5 to 13 g/L, from 5 to 11 g/L, from 5 to 10 g/L, from 5 to 9 g/L, from 5.5 to 8.5 g/L, from 6 to 8.5 g/L, or from 6.5 to 8.5 g/L. In terms of weight percent, the catalyst may comprise less than 2 wt. % Pd, e.g., less than 1.75 wt. %, less than 1.5 wt. %, or less than 1.25 wt. %. In terms of ranges, the catalyst may comprise from 0.1 to <2 wt. % Pd, e.g., from 0.5 to 1.75 wt. %, from 0.75 to 1.5 wt. %, or from 0.8 to 1.15 wt. %.

In some aspects, the inlet, outlet, and/or additional catalyst comprises gold (Au). The inlet catalyst may comprise from 2 to 8 g/L Au, e.g., from 2.5 to 7.5 g/L, from 3 to 7 g/L, from 3 to 6 g/L, from 3 to 5 g/L, from 3.25 to 5 g/L, from 3.5 to 4.75 g/L, or from 3.5 to 4.5 g/L. In some specific aspects, In terms of weight percent, the catalyst may comprise less than 1.5 wt. % Au, e.g., less than 1.25 wt. %, less than 1 wt. %, or less than 0.75 wt. %. In terms of ranges, the catalyst may comprise from 0.1 to 1.5 wt. % Au, e.g., from 0.2 to 1 wt. %, from 0.3 to 0.75 wt. %, or from 0.4 to 0.75 wt. %.

For purposes of calculating the weight percent of components on the catalyst, a density of 630 g/L may be used.

In some aspects, the inlet, outlet, and/or additional catalyst comprises Au and Pd. The ratio of Au to Pd may range from 0.3:1 to 1.5:1, on a g/L basis of Au to Pd, e.g., from 0.4:1 to 1.3:1, from 0.45:1 to 1.2:1, from 0.45:1 to 1:1, from 0.5:1 to 0.75:1, or from 0.5:1 to 0.6:1. In some aspects, Pd is present in a greater amount than Au, in terms of g/L and wt. %.

In some aspects, the inlet, outlet, and/or additional catalyst comprises potassium acetate (KOAc). The KOAc may be present in the greatest amount, as compared to Pd and Au. The KOAc may be present from 25 to 55 g/L, e.g., from 30 to 50 g/L, from 35 to 45 g/L, from 37.5 to 42.5 g/L, or approximately 40 g/L. In terms of weight percent, the KOAc may be present from 40 to 90 wt. %, e.g., from 45 to 85 wt. %, from 50 to 75 wt. %, or from 55 to 70 wt. %.

In some aspects, the inlet, outlet, and/or additional catalyst may contain a refractory support, including a metal oxide such as silica, silica-alumina, titania, or zirconia. In some aspects, the refractory support is silica. The refractory support may then be coated with a layer of catalyst components, e.g., Pd, Au, and KOAc. The thickness of the coating on the refractory support may range from 100 to 300 microns, e.g., from 115 to 200 microns, from 120 to 175 microns, from 125 to 150 microns, from 200 to 300 microns, or from 225 to 275 microns. In some aspects, the thickness of the coating may be selected based on the size of the catalyst.

The inlet catalyst, outlet catalyst, and/or any additional catalyst refractory support may have a cross-sectional shape including, but not limited to, those described above. For example, the inlet catalyst, outlet catalyst, and/or any additional catalyst refractory support may have a pimpled cylindrical, pimpled tubular, pimpled polylobular, pimpled ring, pimpled star, pimpled trilobe, pimpled quadrilobe, pimpled cloverleaf shape, pimpled saddle, pimpled fluted, pimpled ridged, pimpled multi-pointed star, pimpled hollow cylinder, pimpled cogwheel, pimpled spoked wheel, pimpled multi-hole pellet, pimpled monolith, pimpled T-fin shape, pimpled Raschig ring shape, pimpled spherical shape, dimpled cylindrical, dimpled tubular, dimpled polylobular, dimpled ring, dimpled star, dimpled trilobe, dimpled quadrilobe, dimpled cloverleaf shape, dimpled saddle, dimpled fluted, dimpled ridged, dimpled multi-pointed star, dimpled hollow cylinder, dimpled cogwheel, dimpled spoked wheel, dimpled multi-hole pellet, dimpled monolith, dimpled T-fin shape, dimpled Raschig ring shape, dimpled spherical shape, any combination thereof, or other pimpled and/or dimpled shapes. In some aspects, the shape is pimpled spherical and/or dimpled spherical. The size of the spherical shape (excluding the dimples and/or pimples) may range from 1 mm to 10 mm (e.g, 5 to 7 mm), on average. In some aspects, each of the inlet catalyst and outlet catalyst have a different size and a different layer thickness. For example, when the size of the spherical support is 5 mm, the layer is thicker than on a 6 mm or 7 mm shaped support.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included.

As used herein and in the appended claims, the singular forms “a”, “an”, and “the” include plural referents unless the context clearly dictates otherwise. Thus, for example, reference to “a process” includes a plurality of such processes, and so forth.

Also, the words “comprise,” “comprising,” “include,” “including,” and “includes” when used in this specification and in the following claims are intended to specify the presence of stated features, integers, components, or steps, but they do not preclude the presence or addition of one or more other features, integers, components, steps, acts, or groups.

Throughout the application, the term catalyst is used to refer to any or all of the catalyst metal, the catalyst layer, and the entire catalyst structure including the support material.

### EXAMPLES

The following Examples are presented to provide specific representative embodiments of the present invention. The invention is not limited to the specific details as set forth in these Examples.

### Example 1 and Comparative Example 1

Catalyst support materials were prepared using water-based resin (Photopolymer resin: ceramic grey), which is then UV-cured in layers to form the final design. 5 mm spheres were prepared as Comparative Example 1. 5 mm spheres with 2 mm curved pimples located 2.25 mm off the 5 mm sphere center were prepared as Example 1. Once the catalyst support materials were prepared, they were washed with hot water for about 30 seconds and excess moisture was removed. The catalyst support materials were then cured with UV light for 10 minutes in a Creality UV curing and washing machine. The catalyst support materials were tumbled for about 15 to 30 minutes to remove any imperfections remaining.

A tube with a height of 18.25 inches and an inside diameter of 1.33 inches was used to mimic a section of a commercial scale vinyl actetate reactor tube. The catalyst support particles were packed into the tube in average of 1,950 to 3,850 particles depending on their geometry. During the loading, the tube was tapped with a rubber mallet to settle the particles into a dense loading. Instrument air was used to provide flow in the system and was measured by a Coriolis meter. The instrument air enters through the top/inlet of the tube, flows downward over the particles and then exits the tube to atmospheric pressure.

A differential pressure meter was used to measure the pressure difference between the inlet and outlet of the tube, and the average dP was calculated in Excel by importing PI data which recorded the mass flow rate, temperature and dP. A total of 5 trial runs were conducted with each run having a minimum of three different flows to be used as three data points to develop a curve fit of pressure drop per unit length (psi/ft) on the y-axis versus mass flux (lb/ft2·hr) on the x-axis.

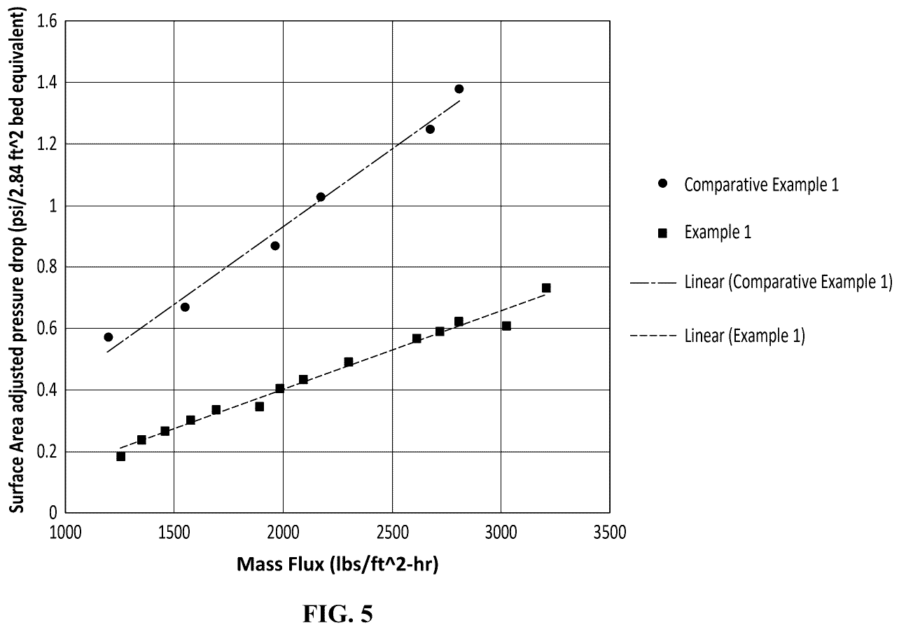

The results for Example 1 (dimpled spheres) and Comparative Example 1 (spheres) of the surface area adjusted pressure drop (psi/2.84 ft² bed equivalent) versus the mass flux (lb/hr·ft²) are shown in FIG. 5. As can be seen in FIG. 5, across the range of mass flux, the surface area adjusted pressure drop for Example 1 is significantly less than Comparative Example 1. If total surface area of catalyst in the tube were held constant, the Example 1 shape would have smaller bed depths and ultimately a lower total pressure drop.

While the invention has been described in detail, modifications within the spirit and scope of the invention will be readily apparent to those of skill in the art. It should be understood that aspects of the invention and portions of various embodiments and various features recited above and/or in the appended claims may be combined or interchanged either in whole or in part. In the foregoing descriptions of the various embodiments, those embodiments which refer to another embodiment may be appropriately combined with other embodiments as will be appreciated by one of ordinary skill in the art. Furthermore, those of ordinary skill in the art will appreciate that the foregoing description is by way of example only, and is not intended to limit the invention. All US patents and publications cited herein are incorporated by reference in their entirety.

What is claimed is:

1. A catalyst for the acetoxylation of olefins, comprising:
a support material having a pimpled external surface; and
a catalytic layer disposed on the external surface, wherein the catalytic layer comprises palladium and gold.

2. The catalyst according to claim 1, wherein the support material is a pimpled sphere.

3. The catalyst according to claim 2, wherein the support material has a diameter from 4 mm to 8 mm, as measured from the longest diameter of the sphere excluding the pimples.

4. The catalyst according to claim 1, wherein the diameter of the pimples, if the pimples were full spheres, is from 0.5 mm to 8 mm.

5. The catalyst according to claim 2, wherein the diameter of the pimples, if the pimples were full spheres, is from 10% to 75% of the diameter of the sphere of the support material without the pimples.

6. The catalyst according to claim 1, wherein the pimples of the support material have a length from their center, if the pimple were a full sphere, to the center of the sphere of the catalyst support material of from 0.1 mm to 5 mm.

7. The catalyst according to claim 1, wherein the support material comprises silica.

8. The catalyst according to claim 7, wherein the support material further comprises alumina.

9. The catalyst according to claim 8, wherein the support material comprises from 60 to 99 wt. % silica and from 1 to 40 wt. % alumina.

10. The catalyst according to claim 1, wherein the catalyst has a void fraction in a 250 mL graduated cylinder (38 mm diameter) of 0.41 or greater.

11. The catalyst according to claim 1, wherein the catalyst layer has a thickness from 100 to 300 μm.

12. The catalyst according to claim 1, wherein the catalytic layer has an average penetration depth into the support material from 115 to 175 microns.

13. The catalyst according to claim 1, wherein the weight ratio of palladium to gold is ≤2:1

14. The catalyst according to claim 1, wherein the weight ratio of palladium to gold is from 1.6:1 to 2:1.

15. The catalyst according to claim 1, wherein the catalyst comprises from 7 to 10 g/L palladium and from 3 to 8 g/L gold.

16. A process for the acetoxylation of an olefin in a gaseous reaction stream containing an olefin, acetic acid, and an oxygen-containing gas, the process comprising:
passing a reaction gas over a reaction tube comprising a catalyst to form an acetoxylated olefin;
wherein the catalyst comprises a support material having a pimpled external surface, and
a catalytic layer disposed on the external surface, wherein the catalytic layer comprises palladium and gold.

17. The process of claim 16, wherein the olefin is ethylene and wherein the selectivity to vinyl acetate monomer is at least 60%.

18. The process of claim 16, wherein the olefin is ethylene and wherein the overall process conversion of ethylene is at least 60%.

19. The process of any of claim 16, wherein the olefin is ethylene and the oxygen conversion is from 35 to 55%.

20. The process of any of claim 16, wherein the olefin is ethylene and the selectivity to carbon dioxide is less than 10%.

21. The process of any of claim 16, wherein the olefin is ethylene and the STY is at least 800 g vinyl acetate monomer/L/hour.

22. The process of any of claim 16, wherein the temperature across the reaction tube is from 145 to 205° C.
